SEQUENCE LISTING (28 sequences)

SEQ ID NO: 1 - protein, 16 aa, Artificial Sequence
  epitope peptide
CGADSYEMEEDGVRKC

SEQ ID NO: 2 - protein, 15 aa, Artificial Sequence
  epitope peptide
CGADSYEMEEDGVRK

SEQ ID NO: 3 - protein, 16 aa, Artificial Sequence
  epitope peptide
CGPDYYEVEEDGIRKC

SEQ ID NO: 4 - protein, 16 aa, Artificial Sequence
  epitope peptide
CNTDTYEVEENGVRKC

SEQ ID NO: 5 - protein, 16 aa, Artificial Sequence
  epitope peptide
CGPDSYEVEEDGVRKC

SEQ ID NO: 6 - protein, 16 aa, Artificial Sequence
  epitope peptide
CSSDSYEVEEDGVRKC

SEQ ID NO: 7 - protein, 16 aa, Artificial Sequence
  epitope peptide
CGADSYEMEEDAVRKC

SEQ ID NO: 8 - protein, 17 aa, Artificial Sequence
  epitope peptide
CPLHNQEVTAEDGTQRC

SEQ ID NO: 9 - protein, 16 aa, Artificial Sequence
  epitope peptide
CPPDKMEVDKNGLKMC

SEQ ID NO: 10 - protein, 16 aa, Artificial Sequence
  epitope peptide
CPSSKMEVEENGIKMC

SEQ ID NO: 11 - protein, 16 aa, Artificial Sequence
  VARIANT 2: Xaa= G, P, N or S
CXXXXXXXXXXXXXXX

SEQ ID NO: 12 - protein, 16 aa, Artificial Sequence
  VARIANT 2: Xaa= G, P, N, Q, S or T
CXXXXXXXXXXXXXXX

SEQ ID NO: 13 - protein, 16 aa, Artificial Sequence
  VARIANT 2: Xaa= G or A
CXXXXXEXXXXGXXXC

SEQ ID NO: 14 - protein, 16 aa, Artificial Sequence
  VARIANT 2: Xaa= G or A
CXXXXXEXXXDGVRKC

SEQ ID NO: 15 - protein, 12 aa, Artificial Sequence
  epitope peptide
CGADSYEMEEDG

SEQ ID NO: 16 - protein, 8 aa, Artificial Sequence
  epitope peptide
CGADSYEM

SEQ ID NO: 17 - protein, 7 aa, Artificial Sequence
  epitope peptide
EEGVRKC

SEQ ID NO: 18 - protein, 134 aa, Mus musculus
MRVLILLWLFTAFPGVLSDVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 19 - protein, 116 aa, Mus musculus
DVQLQESGPSLVKPSQSLSLTCTVTGYSITSDFAWNWIRQFPGNKLEWMGYISYSGNTRYNPSLKSRISITRDTSKNQFFLQLNSVTIEDTATYYCVTAGRGFPYWGQGTLVTVSA

SEQ ID NO: 20 - protein, 128 aa, Mus musculus
MVSTAQFLAFLLLWFPGARCDILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKR

SEQ ID NO: 21 - protein, 108 aa, Mus musculus
DILMTQSPSSMSVSLGDTVSITCHSSQDINSNIGWLQQRPGKSFKGLIYHGTNLDDEVPSRFSGSGSGADYSLTISSLESEDFADYYCVQYAQFPWTFGGGTKLEIKR

SEQ ID NO: 22 - protein, 11 aa, Artificial Sequence
  Heavy chain CDR
GYSITSDFAWN

SEQ ID NO: 23 - protein, 16 aa, Artificial Sequence
  Heavy chain CDR
GYISYSGNTRYNPSLK

SEQ ID NO: 24 - protein, 9 aa, Artificial Sequence
  Heavy chain CDR
VTAGRGFPY

SEQ ID NO: 25 - protein, 11 aa, Artificial Sequence
  Light chain CDR
HSSQDINSNIG

SEQ ID NO: 26 - protein, 7 aa, Artificial Sequence
  Light chain CDR
HGTNLDD

SEQ ID NO: 27 - protein, 9 aa, Artificial Sequence
  light chain CDR
VQYAQFPWT

SEQ ID NO: 28 - protein, 13 aa, Artificial Sequence
  de2-7 junctional peptide
LEEKKGNYVVTDH

(19) United States
(12) Patent Application Publication
(10) Pub. No.: US 2009/0280503 A1
(43) Pub. Date: Nov. 12, 2009

(54) Method for detecting and treating skin disorders

(76) Inventor: James Fiore, Weston, CT (US)

Correspondence Address: KLAUBER & JACKSON, 411 HACKENSACK AVENUE, HACKENSACK, NJ, 07601, US

(21) Appl. No.: 12/310,956

(22) PCT Filed: Sep. 14, 2007
(86) PCT No.: PCT/US2007/019988

§ 371 Date: Jun. 19, 2009

Related U.S. Application Data
(60) Provisional application No. 60/845,099, filed on Sep. 15, 2006.

(51) Int. Cl. A61K 39/395; G01N 33/53; A61K 51/10; A61K 49/16; A61K 49/04; A61P 17/00

(52) U.S. Cl. 435/7.1; 424/145.1; 424/178.1; 424/183.1; 424/1.49; 424/9.1; 424/9.3; 424/9.4

(57) ABSTRACT

The invention provides for methods of detecting and treating diseased tissue, particularly in skin diseases or disorders, such as psoriasis, scleroderma, eczema or atopic dermatitis tissue. The method involves administrating a composition comprising an antibody specific to the diseased tissue to a patient. After administration, the antibody in the composition binds to the exposed cell surface antigen (epitope) and allows the detection and/or disrupts the growth of the diseased tissue. In addition, this invention provides methods of treating psoriasis, scleroderma, eczema or atopic dermatitis by eliciting an immune response in an individual against an antigen which is only exposed to antibody detection in tissues affected by these disorders

### FIELD OF THE INVENTION

The present invention relates generally to methods of detecting and treating diseased tissue, particularly in skin diseases or disorders, such as psoriasis, scleroderma, eczema or atopic dermatitis tissue. The method involves administrating a composition comprising an antibody specific to the diseased tissue to a patient. After administration, the antibody in the composition binds to the exposed cell surface antigen (epitope) and allows the detection and/or disrupts the growth of the diseased tissue. In addition, this invention provides methods of treating psoriasis, scleroderma, eczema or atopic dermatitis by eliciting an immune response in an individual against an antigen which is only exposed to antibody detection in tissues affected by these disorders.

### BACKGROUND OF THE INVENTION

### Skin Diseases Including Psoriasis

The skin is composed of the epidermis, an epithelial layer of ectodermal origin, and the dermis, a layer of connective tissue of mesodermal origin. The epidermis consists mainly of a stratified keratinized epithelium populated primarily by keratinocytes, which are keratinizing epidermal cells. The dermis is composed of the connective tissue that supports the epidermis and binds it to the subjacent layer, known as subcutaneous tissue or hypodermis.

Dermatitis is a superficial inflammation of the skin, characterized by vesicle formation, erythema, edema, oozing, scaling or crusting lesions, and intense itching. Different types of dermatitis can be distinguished: contact dermatitis, caused by irritants in contact with the skin or by non-irritating substances, to which the subject is allergic; atopic dermatitis, characterized by strong itching and chronic course; seborrheic dermatitis, a scaling disease mainly affecting the face and scalp.

One particular form of dermatitis is psoriasis, which is a chronic, inflammatory, hyperproliferative skin disease, characterized by scaling and inflammation that affects 1.5 to 2 percent of the United States population, or almost 5 million people. It occurs in all age groups and about equally in men and women. People with psoriasis suffer discomfort, restricted motion of joints, and emotional distress. When psoriasis develops, patches of skin thicken, redden, and become covered with silvery scales, referred to as plaques. Psoriasis most often occurs on the elbows, knees, scalp, lower back, face, palms, and soles of the feet. The disease also may affect the fingernails, toenails, and the soft tissues inside the mouth and genitalia. About 10 percent of people with psoriasis have joint inflammation that produces symptoms of arthritis. Psoriasis exacts a heavy societal burden in terms of both patient suffering and costs. Annual outpatient treatment of psoriasis was estimated in 1999 to be from $1.6 to $3.2 billion, with over 1.5 million patients seen annually for this disorder by U.S. physicians.

When skin is wounded, a wound healing program is triggered, also known as regenerative maturation. Lesional psoriasis is characterized by cell growth in this alternate growth program. In many ways, psoriatic skin is similar to skin healing from a wound or reacting to a stimulus such as infection, where the keratinocytes switch from the normal growth program to regenerative maturation. Cells are created and pushed to the surface in as little as 2-4 days, and the skin cannot shed the cells fast enough. The excessive skin cells build up and form elevated, scaly lesions. The white scale (called “plaque”) that usually covers the lesion is composed of dead skin cells, and the redness of the lesion is caused by increased blood supply to the area of rapidly dividing skin cells.

The exact cause of psoriasis in humans is not known, although it is generally accepted that it has a genetic component, and a recent study has established that it has an autoimmune component. Whether a person actually develops psoriasis is hypothesized to depend on something “triggering” its appearance. Examples of potential “trigger factors” include systemic infections, injury to the skin (the Koebner phenomenon), vaccinations, certain medications, and intramuscular injections or oral steroid medications.

The chronic skin inflammation of psoriasis is associated with hyperplastic epidermal keratinocytes and infiltrating mononuclear cells, including CD4+ memory T cells, neutrophils and macrophages. Because of this highly mixed inflammatory picture and the resulting complex interrelationships between these different cells, it has been very difficult to dissect the mechanisms that underlie the induction and progression of the disease.

Treatment options currently available to patients suffering from psoriasis include a variety of topical medications, phototherapies, and internal medications. Topical treatments include steroids, coal tar, anthralin, vitamin D3 and analogs, retinoids, and sunshine. Side effects associated with the use of these topical treatments include skin thinning, stretch marks, burns, irritation, and photosensitivity. The use of steroids may also lead to resistance, rendering subsequent steroid treatment ineffective. Phototherapy encompasses the medically supervised administration of ultraviolet light B or psoralen in combination with ultraviolet light A. Long term use of phototherapies may prematurely age the skin and increase the incidence of skin cancers. Internal medications, typically reserved for the most serious cases of psoriasis, include the administration methotrexate, oral retinoids, and cyclosporine. The use of methotrexate requires careful monitoring to avoid liver damage. Use of oral retinoids must be carefully controlled in women because of the potential for severe birth defects. This risk extends for years after the use of the drug has been terminated. Cyclosporine, an immunosuppresant, is reserved for patients that have failed other internal treatments, or for whom the other internal treatments are contraindicated. Rotating between therapies, and combinations of topical medications with phototherapies, has also been found to be useful regimens in the treatment of psoriasis.

Eczema encompasses many kinds of skin problems, including atopic dermatitis, allergic contact eczema, contact eczema, dyshidrotic eczema, neurodermatitis, nummular eczema, seborrheic eczema and statis dermatitis. Eczema (including atopic dermatitis) often has similar overlapping features with psoriasis. For instance, it is often difficult to distinguish based on clinical appearance. They can coexist, or the disease can begin as eczema and over time turn to psoriasis.

There is no cure for psoriasis or eczema, and not all patients respond to or tolerate currently available therapies. New treatment options for psoriasis, with improved efficacy, safety, and side effect profiles, are needed. Similar conditions to psoriasis, eczema, and atopic dermatitis also occur in various domestic animals (mange, etc.). The current invention is felt to encompass all similar diseases in similarly involved species.

Scleroderma (systemic sclerosis) is a type of collagen disease whose major symptoms are fibrosis of skin and organs of viscera such as lung, intestine and the like, and disturbances of peripheral circulation. Symptoms in patients suffering from scleroderma vary and have a wide range from the patients with only extremely light disturbances of circulation who require no treatment at all to the patients who die of respiratory failure, renal failure, cardiac failure or the like within a short period. Scleroderma, or systemic sclerosis, is characterized by deposition of fibrous connective tissue in the skin, and often in many other organ systems. It may be accompanied by vascular lesions, especially in the skin, lungs, and kidneys. The course of this disease is variable, but it is usually slowly progressive. Scleroderma may be limited in scope and compatible with a normal life span. Systemic involvement, however, can be fatal.

Scleroderma is classified as diffuse or limited, on the basis of the extent of skin and internal organ involvement. The diffuse form is characterized by thickening and fibrosis of skin over the proximal extremities and trunk. The heart, lungs, kidneys, and gastrointestinal tract below the esophagus are often involved. Limited scleroderma is characterized by cutaneous involvement of the hands and face. Visceral involvement occurs less commonly.

The anti-EGFR Antibody mAb-806

The monoclonal antibody mAb-806 (class IgG2b) is a murine antibody known for being specific for tumor cells. MAb806 was originally raised to recognize the unique truncation mutant, epidermal growth factor receptor (EGFR) de2-7EGFR or EGFRvIII, using whole cells expressing EGFRvIII mutant as immunogen and has been shown to bind the truncated EGFR present in cancer cells, but not normally expressed wild-type EGFR present on normal cells (Johns T G et al (2002) Int J Cancer 98:398-408). Importantly, the epitope recognized by mAb806 is not accessible in inactive wild-type (wt) EGFR, but is exposed in a transitional form of wt EGFR in cells with overexpression of EGFR, and expression of EGFRvIII (Johns, T G, et al (2004) J Biol Chem 279:30375-30384). The epitope studies are supported by immunohistochemical studies demonstrating that the 806 antibody binds to epitopes present in gliomas, as well as a broad range of epithelial cancers, but not to normal human tissues (Luwor, R B et al (2001) Cancer Res 61:5355-5361; Johns T G et al (2002) Int J Cancer 98:398-408).

These and other preclinical data suggest that mAb806 has a different spectrum of clinical activity and utility and a side effect profile distinct from other anti-EGFR antibodies. MAb806 is distinct from other anti-EGFR antibodies, including antibodies raised against wt EGFR extracellular domain such as cetuximab (also known as antibody 225, U.S. Pat. No. 4,943,533) which generally recognize EGFR, and antibodies raised against the unique de2-7 peptide, which are selective and specific for the truncated receptor and target de2-7 EGFR positive xenografts grown in nude mice (Wikstrand C J et al (1995) Cancer Res 55:3140-3148; Okamoto, S et al (1996) Br J Cancer 73:1366-1372; Hills D et al (1995) Int J Cancer 63:537-543; Reist C J et al (1997) Cancer Res 57:1510-1515; Reist C J et al (1995) Cancer Res 55:4375-4382; U.S. Pat. No. 5,401,828). In xenograft models, mAb806 has exhibited a potent anti-tumor activity with no targeting of normal tissues.

MAb-806 comprises a heavy chain variable region with an amino acid sequence as follows:

| (SEQ ID NO: 18) | |
|---|---|
| MRVLILLWLF TAFPGVLSDV QLQESGPSLV KPSQSLSLTC TVTGYSITSD | 50 |
| FAWNWIRQFP GNKLEWMGYI SYSGNTRYNP SLKSRISITR DTSKNQFFLQ | 100 |
| LNSVTIEDTA TYYCVTAGRG FPYWGQGTLV TVSA | 134 |

MAb-806 comprises a light chain variable region with an amino acid sequences as follows:

| (SEQ ID NO: 20) | |
|---|---|
| MVSTAQFLAF LLLWFPGARC DILMTQSPSS MSVSLGDTVS ITCHSSODIN | 50 |
| SNIGWLQQRP GKSFKGLIYH GTNLDDEVPS RFSGSGSGAD YSLTISSLES | 100 |
| EDFADYYCVQ YAQFPWTFGG GTKLEIKR | |

For both the MAb-806 heavy chain variable region and light chain variable region above, the leader peptide is underlined and the CDR (Complementarity Determining Region) is double underlined. It is understood that the functional mAb-806 antibody does not require, but may still have, the leader peptide. For a detailed description of mAb-806, see, for example PCT applications PCT/US2005/005155 filed Feb. 18, 2005 (published as WO2005081854) or PCT/US02/15185 filed May 13, 2002 (published as WO2002092771) or U.S. application Ser. No. 11/060,646 filed Feb. 17, 2005 (published as US20050255555).

MAb-806 differs from other antibodies that target the de2-7EGFR, in that it does not recognize the unique fusion junction of the truncated receptor de2-7EGFR (Wong A J, et al. Proc Natl Acad Sci USA 1992, 89:2965-2969; Sugawa N. et al. Proc Natl Acad Sci USA 1990, 87:8602-8606; Yamazaki H, et al. Jpn. J. Cancer Res 1990, 81:773-779; Ekstrand A J, et al. Proc Natl Acad Sci USA 1992, 89:4309-4313). MAb 806 does not bind or recognize the unique fusion junction, corresponding to the de2-7 EGFR junctional peptide LEEKKGNYVVTDH (SEQ ID NO:28). Surprisingly, it was found that the binding epitope of mAb-806 exists in both the wild type and truncated de2-7EGFR. Given the ability of mAb-806 to bind both the de2-7EGR and the amplified EGFR, and its absence of binding to normally expressed wild-type receptor, it has been suggested that the mAb-806 epitope is conformationally dependent.

The EGFR binding epitope of mAb 806 has been determined. The epitope receptor peptide, CGADSYEMEEDGVRKC (SEQ ID NO: 1) contains the mAb806 epitope and corresponds to residues 287-302 of EGFR, which form a disulfide-constrained loop in the EGFR.

While it is known that mAb-806 binds to an epitope that is uniquely exposed to antibody binding on tumor cells, there are no reports of mAb-806 showing binding specificity to any other types of cells. Thus, the utility of the mAb-806 outside the field of cancer treatment is unknown.

Epidermal Growth Factor Receptor (EGFR) and the de2-7 EGFR

The EGFR belongs to a family of tyrosine kinase growth factor receptor proteins. The EGFR has long been the subject of investigation, and recently there have been successful structure determination studies performed of the extracellular domains (Ogiso H et al. Cell 2002, 110:775-787; Garrett T P et al. Cell 2002, 110:763-773; Ferguson K M et al Cell 2003, 11:507) and intracellular kinase domain (Stamos J et al J. Biol. Chem. 2002, 277:46265-46272). The EGFR is a cell surface associated molecule, which is activated through binding of highly specific ligand, such as EGF and transforming growth factor alpha (TGF α). After ligand binding, the receptor dimerizes, which results in phosphorylation of the intra-cellular tyrosine kinase region. This leads to downstream signaling, activating a cascade of responses resulting in cell growth and proliferation. The EGFR is normally expressed in the liver and skin, with increased activity often found in solid tumors, such as head and neck, colorectal, pancreas, glioma, bladder and lung, thus making it a useful prognostic marker. Overexpression of the EGFR is often accompanied by increased TGF α production effecting an autocrine loop growth advantage to the tumor.

Furthermore, it was found that the EGFR gene amplification and rearrangement which is observed in some tumors, is often associated with the occurrence of mutant forms of the EGFR (Libermann T A, et al Nature 1985, 313:144-147; Wong A J, Proc Natl Acad Sci USA 1992, 89:2965-2969; Frederick L, et. al Cancer Res 2000, 60:1383-1387). One of the most common mutants is the EGFR variant (EGFRvIII or de2-7EGFR). The de2-7EGFR has an in-frame deletion of 801 base pairs, corresponding to an over-expression of transcripts missing exons 2-7, and a sizeable deletion of amino acid residues 6-273 in the extracellular domain, with a novel glycine inserted at the splice site (Wong A J et al. Proc Natl Acad Sci USA 1992, 89:2965-2969; Sugawa N. et al Proc Natl Acad Sci USA 1990, 87:8602-8606; Yamazaki H. et al Jpn J Cancer Res 1990, 81:773-779; Ekstrand A J et al Proc Natl Acad Sci USA 1992, 89:4309-4313). This truncated form of the EGFR is not dependent on ligand binding, and is constitutively active. The de2-7EGFR is expressed in a large fraction (about 50%) of malignant gliomas and there are also reports linking the de2-7EGFR with breast (27%), ovarian, prostate and lung carcinomas (17%) (Wong A J, et al Proc Natl Acad Sci USA 1992, 89:2965-2969; Garcia d P et al Cancer Res 1993, 53:3217-3220; Wikstrand C J et al Cancer Res 1995, 55:3140-3148; Moscatello D K et al Cancer Res 1995, 55:5536-5539).

Throughout this specification, various patents, patent applications and scientific references are cited to describe the state and content of the art. Those disclosures, in their entireties, are hereby incorporated into the present specification by reference. The citation of references herein shall not be construed as an admission that such is prior art to the present invention.

Therefore, given that there is no cure for the various skin conditions including psoriasis, scleroderma, eczema, or atopic dermatitis and not all patients respond to or tolerate currently available therapies, it should be apparent that there still exists a need in die art for new treatment options which are specific, well-tolerated and efficacious.

### SUMMARY OF THE INVENTION

The present invention relates to a method of treating skin diseases and disorders, including chronic or inflammatory conditions, that cause and include thickening, scaling, hardening, crusting, ulcers, sores, inflammation, or atypical growth of the skin, cutaneous or subcutaneous tissue, epithelial tissue or connective tissue comprising the administration to a patient in need of such treatment of an effective amount of an anti-EGFR antibody which is specific for the diseased or inflamed tissue or which preferentially binds the diseased or inflamed tissue versus normal tissue.

The present invention provides a method of treating psoriasis comprising the administration to a patient in need of such treatment of an effective amount of an antibody which is specific for psoriatic tissue. The present invention provides a method of treating dermatitis comprising the administration to a patient in need of such treatment of an effective amount of an antibody which is specific for the affected tissue. The present invention provides a method of treating scleroderma comprising the administration to a patient in need of such treatment of an effective amount of an antibody which is specific for the affected tissue. This invention also provides for methods of inducing an immune response against psoriasis, scleroderma, eczema, and atopic dermatitis by the administration of therapeutic peptides to a patient.

One aspect of the invention is directed to a method for treating a patient with a skin disorder. The skin disorder may be psoriasis, scleroderma, eczema or atopic dermatitis where psoriatic tissue, scleroderma-affected tissue, eczema tissue or atopic dermatitis tissue appears on the skin of the patient. The method comprises the step of administering a therapeutically effective amount of an antibody or active fragment thereof to the patient to treat the skin disease. The antibody may be any mixture of one or more of the following: (a) a monoclonal antibody 806 (which comprises the amino acid sequences of SEQ ID NO:18 or SEQ ID NO: 19 for the heavy chain variable region and SEQ ID NO:20 or SEQ ID NO:21 for the light chain variable region) or an active fragment thereof; (b) an antibody capable of binding to the epitope on EGFR that is recognized by mAb-806 antibody; (c) an antibody with an epitope specificity to one or more of the “mAb-806 peptide epitopes” (i.e., peptides SEQ ID NO: 1-14. See also the definition of mAb-806 peptide epitopes elsewhere in this disclosure). The antibody may recognize or bind to the same epitope as mAb-806 and/or may compete directly with 806 binding to EGFR or the mAb806 peptide epitope. In the method, one or more of the antibodies (referred to herein as a “therapeutic antibody”) is administered to the patient for the treatment of the skin disorder. The treatment method of the invention reduces at least one symptom of the skin disorder. The one symptom may be, for example, a reduction in psoriatic tissue, a reduction in scleroderma, a reduction in eczema tissue, or a reduction in atopic dermatitis tissue. The symptom may be selected, for example, from one or more of redness, itching, scaling, burning, or thickening of the skin or the psoriatic, scleroderma, eczema or dermatitis tissue.

MAb806 was originally raised to recognize the unique truncation mutant, epidermal growth factor receptor (EGFR) de2-7EGFR or EGFRvIII, and binds the truncated EGFR present in cancer cells, but not normally expressed wild-type EGFR present on normal cells (Johns T G et al (2002) Int J Cancer 98:398-408). Immunohistochemical studies demonstrate that the 806 antibody binds to epitopes present in gliomas, as well as a broad range of epithelial cancers, but not to normal human tissues (Luwor, R B et al (2001) Cancer Res 61:5355-5361; Johns T G et al (2002) Int J Cancer 98:398-408). These and other preclinical data suggest that mAb806 has a different spectrum of clinical activity and utility and a side effect profile distinct from other anti-EGFR antibodies and ligands, which either (i) generally recognize wt and mutant EGFR or (ii) are selective and specific for the truncated mutant EGFR receptor and target de2-7 EGFR. Surprisingly, while 806 does not bind or recognize normal skin tissue, the present invention demonstrates that mAb806 recognizes an EGFR epitope in tissue of skin disorders, including psoriasis, scleroderma, eczema or atopic dermatitis.

It is understood that the term “antibody,” as used in this disclosure, includes a humanized antibody, a single chain antibody, or an engineered antibody. An engineered antibody may be, for example, a single chain polypeptide or two linked polypeptides where the antigen binding domain of the heavy chain and the antigen binding domain of the light chain are placed into at least one of the polypeptide chains. An engineered antibody is also referred to a binding member—which is a polypeptide (or more than one polypeptide which are linked) which is designed to bind an epitope with specificity.

The binding of an antibody to its target antigen is mediated through the complementarily determining regions (CDRs) of its heavy and light chains, with the role of CDR3 being of particular importance. Accordingly, specific binding members based on the CDR3 regions of the heavy or light chain, and preferably both, of mAb806 will be useful specific binding members for in vivo therapy.

It is understood that mAb-806 comprises the sequence of one or more (preferably two) of SEQ ID NO:18, SEQ ID NO:19, SEQ ID NO:20 or SEQ ID NO:21. SEQ ID NO:18 (with leader sequence) and SEQ ID NO:19 (no leader sequence) are the VH amino acid sequence and SEQ ID NO:20 (with leader sequence) SEQ ID NO:21 (no leader sequence) is the VL amino acid sequence.

An engineered antibody, or an antibody capable of binding to the epitope on EGFR that is recognized by mAb-806, may also be a structure or peptide that contains one or more CDR regions as substantially set out as the three CDRs of the mAb-806 heavy chain variable region (GYSITSDFAWN (SEQ ID NO:22), GYISYSGNTRYNPSLK (SEQ ID NO:23) or VTAGRGFPY (SEQ ID NO:24)) or the three CDR regions of the mAb-806 light chain variable region (HSSQDINSNIG (SEQ ID NO:25), HGTNLDD (SEQ ID NO:26) or VQYAQFPWT (SEQ ID NO:27)). In another aspect, the engineered antibody, or an antibody with the same epitope specificity as the mAb-806, may comprise at least the CDR3 region of the mAb-806 heavy chain variable region and/or the CDR3 region of the mAb-806 light chain variable region.

In a preferred embodiment, the therapeutic antibody further comprises a detectable label. Methods of attaching a detectable label to antibodies are well known. The addition of a detectable label allows the monitoring of the antibody after administration to determine its location and clearing rate (the rate at which it becomes ineffective or the rate that it leaves the body by elimination).

While the administration of the therapeutic antibody is sufficient for treatment, the antibody may optionally be conjugated by a chemical bond to one or more moieties that can enhance its effectiveness. These moieties include, at least, a toxin, an immunomodulator, a cytokine, a cytotoxic agent, a chemotherapeutic agent, a radionuclide and a combination of these agents. In an optional step, these moieties may be introduced after the administration of the therapeutic antibody. For example, these moieties may be attached to a second antibody which is specific to the therapeutic antibody. The binding may be performed using known linkage techniques including the use of specific binding pairs. Also, the therapeutic antibody may comprise one member of a specific binding pair. After administration of the therapeutic antibody to a patient, the moiety which is linked to the second member of a specific binding pair may be introduced. The moiety will bind to the therapeutic antibody in vivo based on the specificity of the specific binding pair and exert its effects.

For any of the methods and compositions of the invention, the linkage of any two molecules may involve the use of members of a specific binding pair (also referred to a “binding pair”). A member of a specific binding pair is one of two different molecules, having an area on the surface or in a cavity, which specifically binds to and is thereby defined as complementary with a particular spatial and polar organization of the other molecule. The members of the specific binding pair are referred to as ligand and receptor (antiligand). For example, avidin and biotin form a preferred specific binding pair. The specific binding pair may also be members of an immunological pair such as antigen-antibody, although other specific binding pairs, such as biotin-avidin, hormones-hormone receptors, nucleic acid duplexes, IgG-protein A, DNA-DNA, DNA-RNA, and the like, are not immunological pairs but are specific binding pairs. Other specific binding pairs include streptavidin/biotin, receptor/ligand, poly-His/NTA, antibody/antigen, and the like.

Examples of toxin include: ricin, abrin, ribonuclease, DNase I, Staphylococcal enterotoxin-A, pokeweed antiviral protein, gelonin, diphtherin toxin, Pseudomonas exotoxin, and Pseudomonas endotoxin.

Examples of chemotherapeutic agents includes taxanes, nitrogen mustards, ethylenimine derivatives, alkyl sulfonates, nitrosoureas, triazenes; folic acid analogs, pyrimidine analogs, purine analogs, vinca alkaloids, antibiotics, enzymes, platinum coordination complexes, substituted urea, methyl hydrazine derivatives, azaribine, bleomycin, bryostatin-1, busulfan, carmustine, chlorambucil, cisplatin, CPT-11, cyclophosphamide, cytarabine, dacarbazine, dactinomycin, daunorubicin, dexamethasone, diethylstilbestrol, doxorubicin, ethinyl estradiol, etoposide, fluorouracil, fluoxymesterone, gemcitabine, hydroxyprogesterone caproate, hydroxyurea, L-asparaginase, leucovorin, lomustine, mechlorethamine, medroprogesterone acetate, megestrol acetate, melphalan, mercaptopurine, methotrexate, mithramycin, mitomycin, mitotane, phenyl butyrate, prednisone, procarbazine, semustine streptozocin, tamoxifen, taxol, testosterone propionate, thalidomide, thioguanine, thiotepa, uracil mustard, vinblastine, and vincristine.

Examples of radionuclides include ²²⁵Ac, ¹¹¹Ag, ⁷²As, ⁷⁷As, ²¹¹At, ¹⁹⁸Au, ¹⁹⁹Au, ²¹²Bi, ²¹³Bi, ⁷⁵Br, ⁷⁶Br, ¹¹C, ⁵⁵Co, ⁶²Cu, ⁶⁴CU, ⁶⁷CU, ⁶⁷CU, ¹⁶⁶Dy, ¹⁶⁹Er, ¹⁸F, ⁵²Fe, ⁵⁹Fe, ⁶⁷Ga, ⁶⁸Ga, ¹⁵⁴⁻¹⁵⁸Gd, ¹⁶⁶Ho, ¹²⁰I, ¹²¹I, ¹²³I, ¹²⁴I, ¹²⁵I, ¹³¹I, ¹³¹I, ¹¹⁰In, ¹¹¹In, ¹⁹⁴Ir, ¹⁷⁷Lu, ⁵¹Mn, ⁵²Mn, ⁹⁹Mo, ¹³N, ¹⁵O, ³²P, ³³P, ²¹¹Pb, ²¹²Pb, ¹⁰⁹Pd, ¹⁴⁹Pm, ¹⁴²Pr, ¹⁴³Pr, ²²³Ra, ⁸²Rb, ¹⁸⁶Re, 188Re, ¹⁸⁹Re, ¹⁰⁵Rh, ⁴⁷Sc, ⁷⁵Se, ¹⁵³Sm, ⁸³Sr, ⁸⁹Sr, ¹⁶¹Tb, ⁹⁴Tc, ⁹⁹Tc, ⁸⁶Y, ⁹⁰Y and ⁸⁹Zr.

In a preferred embodiment, the administration is by oral or injectable doses and the amount of radionuclides administered per dose is between 10 and 40 mCi such as between 20-30 mCi.

In a preferred embodiment, the therapeutic antibody administered in the methods of the invention blocks an EGFR to ligand interaction in the psoriatic tissue, the affected scleroderma tissue, eczema tissue, or atopic dermatitis tissue. By blocking the interaction, the excessive growths of these tissues are reduced. Furthermore, with treatment, the psoriatic tissue, the affected scleroderma tissue, eczema tissue, or atopic dermatitis tissue may regress. The regression of these tissues of a skin disorder may cause a reduction of the typical symptoms of skin disorders. These symptoms include, at lease, a plaque phenotype, a guttate phenotype, an inverse phenotype, a pustular phenotype and an erythrodermic phenotype.

In another embodiment, the invention is directed to a method for detecting a skin disorder in a patient. The skin disorder may be manifested by the presences of psoriatic tissue, scleroderma-affected tissue, eczema tissue and atopic dermatitis tissue in the patient. In the method, a mAb-806 antibody or active fragment thereof; an antibody capable of binding to the epitope on EGFR that is recognized by mAb-806 antibody; or an antibody with an epitope specificity to one or more of the peptides SEQ ID NO: 1-14 (or a “mAb-806 peptide epitope” as defined below) is administered to the patient. Following administration, the antibody is bound to the EGFR of a psoriatic tissue, scleroderma-affected tissue, eczema tissue or atopic dermatitis tissue of the patient. Following binding, the antibody is detected. Detection may comprise using a second labeled antibody specific for the administered antibody. Alternatively, detection may comprise detecting a detectable label.

The detectable label may be a fluorescent compound, a chemiluminescent compound, and a bioluminescent compound, or a radionuclide. Examples of fluorescent compounds include fluorescein isothiocyanate, rhodamine, phycoerytherin, phycocyanin, allophycocyanin, o-phthaldehyde and fluorescamine. Examples of chemiluminescent compounds include luminol, isoluminol, aromatic acridinium ester, imidazole, acridinium salt and oxalate ester. Examples of bioluminescent compounds include luciferin, luciferase and aequorin.

Specific binding of the antibody may be determined in vivo by detecting a label from the detectably labeled antibody in said tissue. This may be performed, for example, by using an in vivo imaging method. The in vivo imaging method may be, for example, radionuclide imaging, positron emission tomography, computerized axial tomography, and magnetic resonance imaging.

Alternatively, detection may be performed in vitro. For example, a tissue section may be obtained from the patient and contacted to a labeled antibody which is attached to the detectable label. The tissue section may be examined in vitro for antibody binding by detecting the label.

In one aspect, the present invention includes an assay system which may be prepared in the form of a test kit for the quantitative analysis of the extent of the presence of the skin disease or disorder, particularly psoriasis, scleroderma, dermatitis, eczema and atopic dermatitis. The system or test kit may comprise a labeled component coupling a label to the antibody, and one or more additional immunochemical reagents, at least one of which is a free or immobilized 806 antibody ligand or epitope peptide, capable either of binding with the labeled antibody or its binding partner.

In another aspect, the invention is directed to a method of treating a skin disorder selected from the group consisting of psoriasis, scleroderma, dermatitis, eczema and atopic dermatitis involving the step of administering a peptide selected from any of SEQ ID NO:1-14 (or an “mAb-806 peptide epitope” as defined below) or an immunogenic fragment thereof to the patient. The peptide induces the production of antibodies immunoreactive with the administered peptide or immunogenic fragment thereof. These antibodies also bind an epitope which is present on the psoriatic tissue, affected scleroderma tissue, eczema tissue or atopic dermatitis tissue and whereby said skin disorder is treated.

In this method, the amount of peptide or immunogenic fragment thereof which is administered is between 20 to 1500 milligrams peptide per dose, between 20 to 500 milligrams protein per dose, or between 20 to 100 milligrams protein per dose.

The compositions of use in the invention, including the peptide or immunogenic fragment thereof, may be administered by oral, injectable, or by topical means or routes. Various topical delivery systems are known and can be used to administer a composition of the present invention, e.g., encapsulation in liposomes, microparticles, microcapsules, etc. In preferred embodiments, it is desirable to administer the pharmaceutical compositions of the invention locally to the area in need of treatment; this may be achieved by, for example, and not by way of limitation, topical application, e.g., in conjunction with a wound dressing after surgery, by means of a suppository, or by means of an implant, said implant being of a porous, non-porous, or gelatinous material, including membranes, such as sialastic membranes, or fibers.

For topical administration, the compositions can be formulated in the form of, e.g., an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 4th ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well-known in the art.

In another embodiment, the composition of the invention can be delivered in a vesicle, in particular a liposome (see Langer, Science 249:1527 1533 (1990); Treat et al., in Liposomes in the Therapy of Infectious Disease and Cancer, Lopez-Berestein and Fidler (eds.), Liss, New York, pp. 353 365 (1989); Lopez Berestein, ibid., pp. 317 327; see generally ibid.). In preferred embodiments, the compositions of the present invention do not contain allergenic substances, derivatives from animal sources (such as lanolin, beeswax, animal fat), and certain preservatives (such as parabens, isothiazolones, phenol derivatives, and the like) which can be responsible for or exacerbate allergic contact dermatitis.

The present invention includes an assay system which may be prepared in the form of a test kit for the quantitative analysis of the extent of the presence of the skin disease or disorder, particularly psoriasis, scleroderma, dermatitis, eczema, or atopic dermatitis. The system or test kit may comprise a labeled component coupling a label to the antibody, and one or more additional immunochemical reagents, at least one of which is a free or immobilized 806 antibody ligand or epitope peptide, capable either of binding with the labeled antibody or its binding partner.

Other objects and advantages will become apparent to those skilled in the art from a review of the following description which proceeds with reference to the following illustrative drawings.

### BRIEF DESCRIPTION OF THE DRAWINGS

FIG. 1 depicts tissue sections stained with H& E and visualized following Binding to mAb 806. As can be seen, mAb 806 antibody is confined to the basal layer of the epidermis in psoriatic lesions (bottom left panel, also at higher magnification on the two right panels) and was negative in normal skin (top left panel).

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-II [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

### DEFINITIONS

The amino acid residues described in this specification are preferred to be in the “L” isomeric form. However, residues in the “D” isomeric form can be substituted for any L-amino acid residue, as long as the desired functional property of immunoglobulin-binding is retained by the polypeptide. NH2 refers to the free amino group present at the amino terminus of a polypeptide. COOH refers to the free carboxy group present at tile carboxy tennillus of a polypeptide. In keeping with standard polypeptide nomenclature abbreviations for amino acid residues are as shown in the following Table of Correspondence:

TABLE OF CORRESPONDENCE

| SYMBOL | | |
|---|---|---|
| 1 Letter | 3 Letter | AMINO ACID |
| Y | Tyr | tyrosine |
| G | Gly | glycine |
| F | Phe | phenylalanine |
| M | Met | methionine |
| A | Ala | alanine |
| S | Ser | serine |
| I | Ile | isoleucine |
| L | Leu | leucine |
| T | Thr | threonine |
| V | Val | valine |
| P | Pro | proline |
| K | Lys | lysine |
| H | His | histidine |
| Q | Gln | glutamine |
| E | Glu | glutamic acid |
| W | Trp | tryptophan |
| R | Arg | arginine |
| D | Asp | aspartic acid |
| N | Asn | asparagine |
| C | Cys | cysteine |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The above Table is presented to correlate the three-letter and one-letter notations which may appear alternately herein.

As used herein, the term “engineered antibody” encompasses all biochemically or recombinantly produced functional derivatives of antibodies. A protein is a functional derivative of an antibody if it has at least one antigen binding site (ABS) or a complementarity-determining region (CDR) that when combined with other CDR regions (on the same polypeptide chain or on a different polypeptide chain) can form an ABS. The definition of engineered antibody would include, at least, recombinant antibodies, tagged antibodies, labeled antibodies, Fv fragments, Fab fragments, recombinant (as opposed to natural) multimeric antibodies, single chain antibodies, and other higher multimeric forms of antibodies such as linked antibodies.

As used herein, the term “single-chain antibody (scFv),” refers to engineered antibody constructs prepared by isolating the binding domains (both heavy and light chain) of a binding antibody, and supplying a linking moiety which permits preservation of the binding function. This forms, in essence, a radically abbreviated antibody, having only the variable domain necessary for binding the antigen. Determination and construction of single chain antibodies are described in many prior publications including U.S. Pat. No. 4,946,778; Bird et al., Science 242:423 (1988) and Huston et al., Proc. Nat'l Acad. Sci. USA 85:5879 (1988).

The term “humanized” means that at least a portion of the framework regions of an immunoglobulin or engineered antibody construct (including the fusion molecules of this invention that comprise an immunoglobulin or engineered antibody) is derived from human immunoglobulin sequences. It should be clear that any method to humanize antibodies or antibody constructs, as for example by variable domain resurfacing as described by Roguska et al., (1994) Proc. Natl. Acad. Sci. USA 91: 969-973 would be applicable to the fusion molecules of this invention. Alternatively, CDR grafting (also called CDR shuffling) or reshaping as reviewed by Hurle and Gross ((1994) Curr. Opin. Biotech. 5:428-433), can be used. Manipulation of the complementarity-determining regions (CDR) is a way of achieving humanized antibodies. The use of antibody components derived from humanized monoclonal antibodies obviates potential problems associated with the immunogenicity of murine constant regions. General techniques for cloning murine immunoglobulin variable domains are described, for example, by the publication of Orlandi et al., Proc. Nat'l Acad. Sci. USA 86: 3833 (1989). Techniques for producing humanized monoclonal antibodies (MAbs) are described, for example, by Jones et al., Nature 321: 522 (1.986), Riechmann et al., Nature 332: 323 (1.988), Verhoeyen et al., Science 239: 1534 (1988), Carter et al., Proc. Nat'l Acad. Sci. USA 89: 4285 (1992), Sandhu, Crit. Rev Biotech. 12: 437 (1992), Singer et al., J. Immun. 150: 2844 (1993), Winter & Milstein, Nature 349:293 (1991).

It is understood that any methods and compositions of the invention may be used to treat any animal including humans. In preferred embodiments, any antibodies used for any purpose in humans are partially humanized (comprising mostly human sequence) or completely humanized. A completely humanized antibody may be produced, for example, by the production of an antibody in humans. It should be noted that a human subject is not needed for production of human antibodies. For example, human antibodies may be produced from an expression library of human antibodies or from a transgenic animal with human immunoglobulin genes. Other methods of producing human antibodies without the use of human subjects are known.

The term “treating” in its various grammatical forms in relation to the present invention refers to preventing, curing, reversing, attenuating, alleviating, minimizing, mitigating, suppressing or halting the deleterious effects of a disease state, disease progression, disease causative agent (e.g., bacteria or viruses) or other abnormal condition. Because some of the inventive methods involve the physical removal of the etiological agent, the artisan will recognize that they are equally effective in situations where the inventive compound is administered prior to, or simultaneous with, exposure to the etiological agent (prophylactic treatment) and situations where the inventive compounds are administered after (even well after) exposure to the etiological agent.

Unless otherwise noted, use of the term “antibody” or “immunoglobulin” herein will be understood to include antibody fragments and functional derivatives (i.e., engineered antibody) thereof. Antibodies can be whole immunoglobulin of any class, e.g., IgG, IgM, IgA, IgD, IgE, or hybrid antibodies with dual or multiple antigen or epitope specificities, or fragments, e.g., F(ab′)2, F(ab)2, Fab′, Fab1 and the like, including hybrid fragments. Functional derivatives include engineered antibodies.

According to the specific case, the “therapeutically effective amount” of an antibody should be determined as being the amount sufficient to improve the symptoms of the patient in need of treatment or at least to partially arrest the disorder (disease) and its complications. Amounts effective for such use will depend on the severity of the disorder and the general state of the patient's health. Single or multiple administrations may be required depending on the dosage and frequency as required and tolerated by the patient.

As used herein, the term “a method to detect” refers to any assay (including immunoassays and calorimetric assays) known in the art for the measurement of a detectable label. These assays include, at least, assays utilizing e biotin and avidin or streptavidin, ELISAs, immunoprecipitation, immunohistochemical techniques and agglutination assays. A detailed description of these assays is given in, e.g., WO 96/13590 to Maertens & Stuyver. The term “biological sample” relates to any possible sample taken from an animal (including humans), such as blood (which also encompasses platelets, red blood cells, and serum and plasma samples), sputum, cerebrospinal fluid, urine, saliva, sweat, lymph or any possible histological section, and other body fluid. Detection may also include methods of imaging a lesion, such as with immunoscintigraphy, computed tomography (CT), ultrasonography, X-rays, and the like.

The terms “binding specificity,” “specifically binds to” or “specifically immunoreactive with,” when referring to a protein or antibodies of the invention, refers to a binding reaction which is determinative of the presence of the protein or carbohydrate in the presence of a heterogeneous population of proteins and other biological components. Thus, under designated immunoassay conditions, the specified fusion polypeptide binds to a particular protein or carbohydrate, or other moiety, and does not bind in a significant amount to other proteins or carbohydrates, or other moieties present in the sample. Specific binding to a fusion polypeptide under such conditions may require a fusion polypeptide selected for its specificity towards a particular protein or carbohydrate, or other moiety. For example, fusion polypeptides directed to any one of SEQ ID NO:1-14 may be selected to provide fusion polypeptides that are specifically immunoreactive with any one of SEQ ID NO:1-14 and not with other proteins. A variety of immunoassay formats may be used to select fusion polypeptide specifically immunoreactive with a particular protein or carbohydrate. As one example, solid-phase ELISA immunoassays are routinely used to select antibodies specifically immunoreactive with a protein or carbohydrate, or other moiety. See Harlow & Lane, Antibodies, A Laboratory Manual, Cold Spring Harbor Publication, New York (1988) for a description of immunoassay formats and conditions that can be used to determine specific immunoreactivity.

The terms “psoriasis tissue” and “psoriatic tissue” have the same meaning. The invention is based, in part, on the surprising discovery that an epitope recognized by mAb-806, which is undetectable in normal skin using mAb-806, becomes detectable in psoriasis tissue, affected scleroderma tissue, eczema tissue or atopic dermatitis tissue. MAb-806 binds to and recognizes an EGFR epitope which is present in skin tissue from psoriasis patients, but absent in normal skin. While normal skin or normal human epidermis has detectable EGF receptors, particularly in mitotically active basal keratinocytes (Nanney L B et al (1984) J Invest Dermatol 83:385-393), and Neirinckx suggests topical treatment of psoriasis with EGF (U.S. Pat. No. 7,015,199), which will affect both normal and diseased tissue, a unique and distinct form of EGFR has not been previously recognized or identified in this skin disorder. Antibody mAb806, or other antibodies or antibody fragments which recognize the particular 806 epitope or 806 epitope peptide(s), provide a specific and targeted treatment for psoriatic and other skin disorder tissue or lesions.

### I. Treatments

This invention provides a method for treating a patient with psoriasis tissue, scleroderma tissue, eczema tissue or atopic dermatitis tissue, or a combination of these tissues, by administering an antibody (including an engineered antibody) which is capable of specifically binding a cell surface antigen which is specific to these tissues to a patient. We have found, surprisingly, that the following three types of antibodies (referred to herein as “therapeutic antibodies” can bind selectively to psoriasis, eczema, or atopic dermatitis tissues: (1) the murine monoclonal antibody mAb-806 antibody or a derivative thereof; (2) an antibody with the same epitope specificity as the mAb-806, or (3) an antibody which are specific for the mAb-806 peptide epitopes.

The terms “MAb806,” “mAb-806”, “806 antibody”, “antibody 806”, “Ab806”, and any variants not specifically listed, may be used herein interchangeably, and as used throughout the present application and claims refer to antibodies, active fragments thereof, monomers thereof, recombinant derivatives thereof, and extends to those proteins having the amino acid sequence data described herein and presented in any of SEQ ID NOS:18-21 or fragments thereof, and the profile of activities set forth herein and in the Claims. The terms also include a structure that contains one or more CDR regions of antibody 806 as substantially set out as the three CDRs of the mAb-806 heavy chain variable region (GYSITSDFAWN (SEQ ID NO:22), GYISYSGNTRYNPSLK (SEQ ID NO:23) or VTAGRGFPY (SEQ ID NO:24)) or the three CDR regions of the mAb-806 light chain variable region (HSSQDINSNIG (SEQ ID NO:25), HGTNLDD (SEQ ID NO:26) or VQYAQFPWT (SEQ ID NO:27)). Accordingly, proteins displaying substantially equivalent or altered activity are likewise contemplated. These modifications may be deliberate, for example, such as modifications obtained through site-directed mutagenesis, or may be accidental, such as those obtained through mutations in hosts that are producers of the complex or its named subunits. Also, the terms “MAb806,” “mAb-806”, “806 antibody”, “antibody 806”, “Ab806” are intended to include within their scope proteins specifically recited herein as well as all substantially homologous analogs and allelic variations.

MAb806 was originally raised to recognize the unique truncation mutant, epidermal growth factor receptor (EGFR) de2-7EGFR or EGFRvIII, and binds to epitopes present in gliomas, as well as a broad range of epithelial cancers, but not to normal human tissues (Luwor, R B et al (2001) Cancer Res 61:5355-5361; Johns T G et al (2002) Int J Cancer 98:398-408). MAb806 has a different spectrum of clinical activity and utility and a side effect profile distinct from other anti-EGFR antibodies and ligands, which either (i) generally recognize both or all of wt and mutant EGFR or (ii) are selective and specific for the truncated mutant EGFR receptor and target de2-7 EGFR. Surprisingly, while 806 does not bind or recognize normal skin tissue, the present invention demonstrates that mAb806 recognizes an EGFR epitope in tissue of skin disorders, including psoriasis, scleroderma, eczema or atopic dermatitis.

The EGFR binding epitope of antibody mAb 806 has been determined. The epitope receptor peptide, CGADSYEMEEDGVRKC (SEQ ID NO: 1) contains the mAb806 epitope and corresponds to residues 287-302 of EGFR, which form a disulfide-constrained loop in the EGFR (Chao et al (2004) J Mol Biol 342(2):539-550; PCT application PCT/US2005/005155 filed Feb. 18, 2005 (published as WO2005081854); U.S. application Ser. No. 11/060,646 filed Feb. 17, 2005 (published as US20050255555). Natural alleles and variants of this loop epitope peptide sequence in various mammalian EGFRs and in a corresponding loop sequence of EGF family members EGFR, ErbB2, ErbB3 and ErbB4 are encompassed and included in “mAb-806 peptide epitopes”.

“mAb-806 peptide epitopes” are defined as and include SEQ ID NO: 1 to SEQ ID NO:14 and preferred embodiments of these peptides as defined below:

| | |
|---|---|
| CGADSYEMEEDGVRKC. | (SEQ ID NO: 1) |
| CGADSYEMEEDGVRK. | (SEQ ID NO: 2) |
| CGPDYYEVEEDGIRKC. | (SEQ ID NO: 3) |
| CNTDTYEVEENGVRKC. | (SEQ ID NO: 4) |
| CGPDSYEVEEDGVRKC. | (SEQ ID NO: 5) |
| CSSDSYEVEEDGVRKC. | (SEQ ID NO: 6) |
| CGADSYESYEMEEDAVRKC. | (SEQ ID NO: 7) |
| CPLHNQEVTAEDGTQRC. | (SEQ ID NO: 8) |
| CPPDKMEVDKNGLKMC. | (SEQ ID NO: 9) |
| CPSSKMEVEENGIKMC. | (SEQ ID NO: 10) |
| C X₁ X₂ X₃ X₄ X₅ X₆ X₇ X₈ X₉ X₁₀ X₁₁ X₁₂ X₁₃ X ₁₄ X₁₅ | (SEQ ID NO: 11) |

where each Xₙ residue can be independently selected as follows:

X₁ is G, P, N or S;

X₂ is A, P, T, S or L;

X₃ is D, H or S;

X₄ is S, Y, T, N or K;

X₅ is Y, Q or M;

X₆ is M or V;

X₇ is E, T or D;

X₈ is A or none;

X₉ is E or K;

X₁₀ is D or N;

X₁₁ is G or A;

X₁₂ is V, I, L or T;

X₁₃ is R, Q or K;

X₁₄ is R, K or M;

X₁₅ is C or none.

C X₁ X₂ X₃ X₄ X₅ X₆ X₇ X₈ X₉ X₁₀ X₁₁ X₁₂ X₁₃ X₁₄ X₁₅ (SEQ ID NO: 12) where each Xₙ residue can be independently selected as follows::

X₁ is G, P, N, Q, S or T

X₂ is A, P, T, S, L, M, V, I or P

X₃ is D, E, H, R, K, S or T

X₄ is S, Y, F, W, T, N, Q, K or R

X₅ is Y, F, W, Q, N, M, V, A, L, I or P

X₆ is M, V, A, L, I or P

X₇ is E, D, T or S

X₈ is A, V, L, I, P, M or none

X₉ is D, E, K or R

X₁₀ is D, E, N or Q

X₁₁ is G, A, M, V, L, I or P

X₁₂ is V, I, L, M, A, P, S or T

X₁₃ is R, K, H, Q or N

X₁₄ is R, K, H, M, A, V, L, I or P

X₁₅ is C or none.

C X₁ X₂ X₃ X₄ X₅ E X₆ X₇ X₈ X₉ G X₁₀ X₁₁ X₁₂ C (SEQ ID NO: 13) where each Xₙ residue can be independently selected as follows:

X₁ is G or A

X₂ is A or K

X₃ is D or A

X₄ is S or A

X₅ is Y or A

X₆ is M or A

X₇ is E or A

X₈ is E or A

X₉ is D or A

X₁₀ is V, A or K

X₁₁ is R or A

X₁₂ is K or A.

C X₁ X₂ X₃ X₄ X₅ E X₆ X₇ Xs DGVRKC (SEQ ID NO:14) where each Xₙ residue can be independently selected as follows:

X₁ is G or A

X₂ is A or K

X₃ is D or A

X₄ is S or A

X₅ is Y or A

X₆ is M or A

X₇ is E or A

X₈ is E or A.

Mutations can be made in the mAb-806 antibody(ies) or active fragments thereof (encompassing and including SEQ ID NOS:18-21 or fragments thereof), or the mAb-806 peptide epitopes (encompassing and including SEQ ID NOS:1-14) or proteins thereof such that one or more amino acid is replaced or substituted with a different amino acid. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include antibodies, fragments thereof or epitope peptides or proteins thereof containing one or more conservative changes, and/or non-conservative changes, or combinations thereof, which do not significantly alter the activity or binding characteristics of the resulting protein (e.g., antibody, antibody fragment, or peptide).

The 806 antibody of use in the invention includes antibodies as set out in SEQ ID NOS:18-21, active fragments thereof, monomers thereof, recombinant derivatives thereof, or fragments thereof, include an antibody or polypeptide that contains one or more CDR regions of antibody 806 as substantially set out as the three CDRs of the mAb-806 heavy chain variable region (GYSITSDFAWN (SEQ ID NO:22), GYISYSGNTRYNPSLK (SEQ ID NO:23) or VTAGRGFPY (SEQ ID NO:24)) or the three CDR regions of the mAb-806 light chain variable region (HSSQDINSNIG (SEQ ID NO:25), HGTNLDD (SEQ ID NO:26) or VQYAQFPWT (SEQ ID NO:27)). Fragments of the Mab-806 peptide epitopes of SEQ ID NOS: 1-14 may act as Mab-806 peptide epitopes, and include any of SEQ ID NOS: 15, 16 and 17.

The following provides examples of various groupings of amino acids:

Amino acids with nonpolar R groups: Alanine, Valine, Leucine, Isoleucine, Proline Phenylalanine, Tryptophan, Methionine

Amino acids with uncharged polar R groups: Glycine, Serine, Threonine, Cysteine, Tyrosine, Asparagine, Glutamine

Amino acids with charged polar R groups (negatively charged at Ph 6.0): Aspartic acid, Glutamic acid

Basic amino acids (positively charged at pH 6.0): Lysine, Arginine Histidine (at pH 6.0)

Another grouping may be those amino acids with phenyl groups: Phenylalanine, Tryptophan, Tyrosine

Another grouping may be according to molecular weight (i.e., size of R groups):

| | |
|---|---|
| Glycine | 75 |
| Alanine | 89 |
| Serine | 105 |
| Proline | 115 |
| Valine | 117 |
| Threonine | 119 |
| Cysteine | 121 |
| Leucine | 131 |
| Isoleucine | 131 |
| Asparagine | 132 |
| Aspartic acid | 133 |
| Glutamine | 146 |
| Lysine | 146 |
| Glutamic acid | 147 |
| Methionine | 149 |
| Histidine (at pH 6.0) | 155 |
| Phenylalanine | 165 |
| Arginine | 174 |
| Tyrosine | 181 |
| Tryptophan | 204 |

Particularly preferred substitutions are:

Lys for Arg and vice versa such that a positive charge may be maintained;

Glu for Asp and vice versa such that a negative charge may be maintained;

Ser for Thr such that a free —OH can be maintained; and

Gln for Asn such that a free NH₂ can be maintained.

Amino acid substitutions may also be introduced to substitute an amino acid with a particularly preferable property. For example, a Cys may be introduced a potential site for disulfide bridges with another Cys. A His may be introduced as a particularly “catalytic” site (i.e., His can act as an acid or base and is the most common amino acid in biochemical catalysis). Pro may be introduced because of its particularly planar structure, which induces β-turns in the protein's structure.

Two amino acid sequences are “substantially homologous” when at least about 70% of the amino acid residues (preferably at least about 80%, and most preferably at least about 90 or 95%) are identical, or represent conservative substitutions.

The phrase “pharmaceutically acceptable” refers to molecular entities and compositions that are physiologically tolerable and do not typically produce an allergic or similar untoward reaction, such as gastric upset, dizziness and the like, when administered to a human.

The administration of these antibodies can be used to treat a patient suffering from the presence of psoriasis tissue, eczema tissue or atopic dermatitis tissue.

This invention provides a method of inhibiting the growth of psoriasis tissue, scleroderma tissue, dermatitis tissue, eczema tissue or atopic dermatitis tissue in an animal which comprises administering to the animal an effective inhibiting amount of a pharmaceutical composition comprising one or more of the therapeutic antibodies described above and a pharmaceutically acceptable carrier. For the purposes of this invention, an “effective inhibiting amount” of a pharmaceutical composition is any amount of the pharmaceutical composition which is effective to bind to a protein on the surface of psoriasis tissue, scleroderma tissue, dermatitis tissue, eczema tissue or atopic dermatitis tissue. This effective inhibiting amount may easily be determined by an ordinary skilled practitioner using experiments well known in the art. One such experimental approach is by titration. For example, a starting amount of antibody may be administered to a patient and the patient may be observed for a period of time, such as two weeks, to determine if there is a regression of psoriasis tissue, scleroderma tissue, eczema tissue or atopic dermatitis tissue. If there is regression, the treatment may be continued. If there is insufficient or no regression, the dosage of antibodies may be increased.

The therapeutic antibodies of this invention may be combined with other treatments of psoriasis, scleroderma, eczema or atopic dermatitis, e.g., an antibiotic, immune suppressive therapeutic, immune adjuvant, analgesic, anti-inflammatory drug and the like. See, e.g., the Physician's Desk Reference, both prescription and non-prescription compendiums.

Preferred combination therapies include the therapeutic antibodies with various anti-inflammatory agents, such as topical, transdermal, or systemic steroids or corticosteroids. Systemic, topical, transdermal, or systemic retinoid or retinoid-like compounds, or vitamin D analogs, may be administered with the therapeutic antibodies. Alternatively, various forms of UV light may be used in combination with these therapeutics, e.g., ultraviolet A, ultraviolet B, or narrow bands of UVB.

To prepare pharmaceutical or sterile compositions including the therapeutic antibodies, the antibodies are admixed with a pharmaceutically acceptable carrier or excipient which is preferably inert. Preparation of such pharmaceutical compositions is known in the art, see, e.g., Remington's Pharmaceutical Sciences and U.S. Pharmacopeia: National Formulary, Mack Publishing Company, Easton, Pa. (1984). Typically, therapeutic compositions are sterile.

When administered parenterally the therapeutic antibodies will be formulated in a unit dosage injectable form (solution, suspension, emulsion) in association with a pharmaceutically acceptable parenteral vehicle. Such vehicles are inherently nontoxic and nontherapeutic. The therapeutic antibodies may be administered in aqueous vehicles such as water, saline, or buffered vehicles with or without various additives and/or diluting agents. Such a composition can be useful for subcutaneous (SQ), intradermal (ID), or intramuscular (IM) injection. The proportion of therapeutic entity and additive can be varied over a broad range so long as both are present in effective amounts.

Selecting an administration regimen for therapeutic antibodies depends on several factors, including the serum or tissue turnover rate of the therapeutic, the immunogenicity of the therapeutic, or the accessibility of the target cells. Preferably, an administration regimen maximizes the amount of therapeutic delivered to the patient consistent with an acceptable level of side effects. Accordingly, the amount of therapeutic delivered depends in part on the particular agonist or antagonist and the severity of the condition being treated. Guidance in selecting appropriate doses of antibodies is found in the literature on therapeutic uses, e.g. Bach et al., chapter 22, in Ferrone, et al. (eds. 1985) Handbook of Monoclonal Antibodies Noges Publications, Park Ridge, N.J.; and Russell, pgs. 303-357, and Smith et al., pgs. 365-389, in Haber, et al. (eds. 1977) Antibodies in Human Diagnosis and Therapy Raven Press, New York, N.Y.

Determination of the appropriate dose is made by the clinician, e.g., using parameters or factors known in the art to affect treatment or predicted to affect treatment. Generally, the dose begins with an amount somewhat less than the optimum dose and it is increased by small increments thereafter until the desired or optimum effect is achieved relative to any negative side effects.

The total weekly dose ranges for therapeutic antibodies range generally from about 1 ng, more generally from about 10 ng, typically from about 100 ng; more typically from about 1 μg, more typically from about 10 μg, preferably from about 100 μg, and more preferably from about 1 mg per kilogram body weight. Although higher amounts may be more efficacious, the lower doses typically will have fewer adverse effects. Generally the range will be less than 100 mg, preferably less than about 50 mg, and more preferably less than about 25 mg per kilogram body weight.

The present invention also provides for administration of therapeutic antibodies in combination with known therapies, e.g., steroids, particularly glucocorticoids, which alleviate the symptoms associated with excessive inflammatory responses. Daily dosages for glucocorticoids will range from at least about 1 mg, generally at least about 2 mg, and preferably at least about 5 mg per day. Generally, the dosage will be less than about 100 mg, typically less than about 50 mg, preferably less than about 20 mg, and more preferably at least about 10 mg per day. In general, the ranges will be from at least about 1 mg to about 100 mg, preferably from about 2 mg to 50 mg per day.

The phrase “effective amount” means an amount sufficient to effect a desired response, or to ameliorate a symptom or sign of the skin condition.

Any of the methods of this disclosure may be used to treat any patient. A patient may be any animal. Preferably, the patient is a mammal. More preferably, the patient is a mammal such as mice, rats, cats, dogs, pigs, horses, cows, goats, commercially valuable livestock, and primates, including humans. An effective amount for a particular patient may vary depending on factors such as the condition being treated, the overall health of the patient, the method, route, and dose of administration and the severity of side affects. Preferably, the effect will result in a change in quantitation of at least about 10%, preferably at least 20%, 30%, 50%, 70%, or even 90% or more. The quantitation may be, for example, the amount of psoriasis tissue, eczema tissue, or atopic dermatitis tissue present.

### II. Immunoassays

Immunoassays are valuable in diagnosing psoriasis, scleroderma, eczema, or atopic dermatitis. Qualitative or quantitative measurement of the mAb-806 antigen can be performed by a variety of immunoassay methods for a diagnosis of psoriasis, scleroderma, eczema, or atopic dermatitis or an early stage of these conditions where external symptoms are absent, For a review of immunological and immunoassay procedures in general, see Stites and Terr (eds. 1991) Basic and Clinical Immunology (7th ed.). Moreover, the immunoassays of the present invention can be performed in many configurations, which are reviewed extensively in, e.g., Maggio (ed. 1980) Enzyme Immunoassay CRC Press, Boca Raton, Fla.; Tijan (1985) “Practice and Theory of Enzyme Immunoassays,” Laboratory Techniques in Biochemistry and Molecular Biology, Elsevier Science Publishers B.V., Amsterdam; Harlow and Lane Antibodies: A Laboratory Manual, supra; Chan (ed. 1987) Immunoassay: A Practical Guide Academic Press, Orlando, Fla.; Price and Newman (eds. 1991) Principles and Practice of Immunoassays Stockton Press, NY; and Ngo (ed. 1988) Non-isotopic Immunoassays Plenum Press, NY.

In particular, the present invention provides various skin related diseases as conditions susceptible to analysis or diagnosis by evaluating the presence of mAb-806 antigens. For example, the presence or severity of psoriasis, scleroderma, eczema, or atopic dermatitis can be evaluated by presence, concentration or total amount of mAb-806 antigens bearing cells present. Prophylactic or therapeutic downregulation of mAb-806 antigens may be useful to treat or prevent these and similar or related skin diseases. The present invention includes an assay system which may be prepared in the form of a test kit for the quantitative analysis of the extent of the presence of the skin disease or disorder, particularly psoriasis, scleroderma, dermatitis, eczema and atopic dermatitis. The system or test kit may comprise a labeled component coupling a label to the antibody, and one or more additional immunochemical reagents, at least one of which may be a free or immobilized 806 antibody ligand or epitope peptide, capable either of binding with the labeled antibody or its binding partner.

Immunoassays for measurement of mAb-806 antigens can be performed by a variety of methods known to those skilled in the art. In brief, immunoassays to measure the protein can be either competitive or noncompetitive binding assays. In competitive binding assays, the sample to be analyzed competes with a labeled analyte for specific binding sites on a capture agent bound to a solid surface. Preferably the capture agent is an antibody specifically reactive with mAb-806 antigen proteins. The concentration of labeled analyte bound to the capture agent is inversely proportional to the amount of free analyte present in the sample.

mAb-806 antigens may also be determined by a variety of noncompetitive immunoassay methods. For example, a two-site, solid phase sandwich immunoassay may be used. In this type of assay, a binding agent for the mAb-806 antigens, e.g., mAb-806 is attached to a solid support. A second protein binding agent, which may also be an antibody, and which binds the mAb-806 antigen at a different site, e.g., an EGRF antibody, is labeled. After binding at both sites on the protein has occurred, the unbound labeled binding agent is removed and the amount of labeled binding agent bound to the solid phase is measured. The amount of labeled binding agent bound is directly proportional to the amount of protein in the sample.

Western blot analysis can be used to determine the presence of mAb-806 antigens in a sample. Electrophoresis is carried out, for example, on a tissue sample suspected of being a psoriasis, scleroderma, eczema, or atopic dermatitis tissue. Following electrophoresis to separate the proteins, and transfer of the proteins to a suitable solid support, e.g., a nitrocellulose filter, the solid support is incubated with an antibody reactive with mAb-806 antigens. This antibody (e.g., mAb-806) may be labeled, or alternatively may be detected by subsequent incubation with a second labeled antibody that binds the primary antibody.

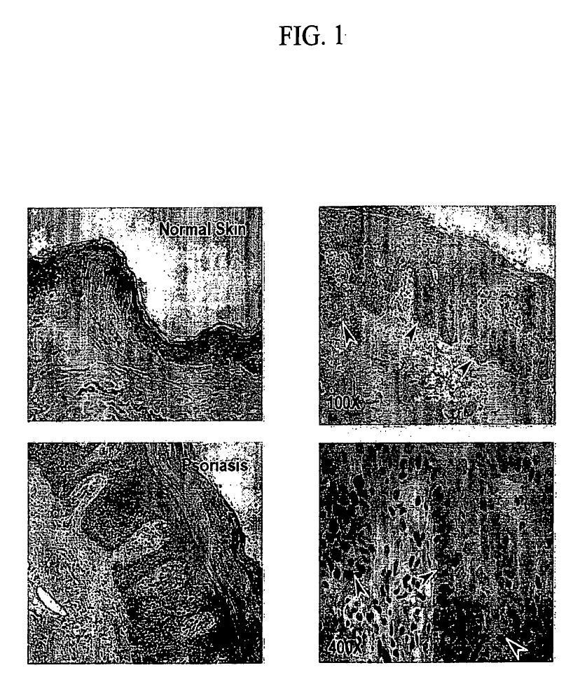

In a preferred method of diagnosis, the antigen detection may be performed using immunohistochemistry (See, e.g., Example 1 and FIG. 1). Methods for immunohistochemistry are known. Cells and tissue biopsy samples which may be used to prepare the sample for immunohistochemistry include cytospins, cell pellets, paraffin-embedded sections, or other specimens that have been frozen or formalin-fixed, and the like. The number of cells involved will usually be at least about 1,000. For diagnosis of a patient, usually a biopsy will be the source of the tissue. Depending upon the manner of preparation of the sample, there may be differences in the observed signal. Therefore, one method of preparing the sample will be preferred over another, depending upon the level of signal over the range of interest. For example, paraffinized tissue may provide for lower levels of immunostaining than frozen tissue. For tissue, usually the sample will have an area of equivalent size to from about 0.2 cm to 2.0 cm in diameter. The methods of preparing the sample are well known and have been amply described in a wide variety of texts and papers. See, for example, Theory and Practice of Histotechnology by Dezna Sheehan and Barbara Hrapchak; and Diagnostic Cytopathology by Leopold Koss.

The prepared sample may then be combined with a labeled binding composition comprising a specifically binding probe (antibody) for immunohistochemical detection. Various labels may be employed which provide for spectrophotometric detection, particularly fluorescers, or enzymes which produce a product which absorbs light or fluoresces, preferably fluoresces. A wide variety of labels are known which provide for strong signals in relation to a single binding event. Fluorescent molecules which may be used include intercalated staining dyes in DNA chains, such as are described in U.S. Pat. No. 5,321,130, phycoerythrins, fluorescein, rhodamine, Texas red, or enzymes which provide for colored dyes or fluorescers, which enzymes include hydrolases, e.g. phosphatases and glycosidases, oxidoreductases, such as peroxidases, oxidases, NADH dependent enzymes, etc. Enzyme dye combinations include alkalaine phosphatase/CAS red, hroseradish peroxidaseldiaminobenzidine, amino ethylcarbazole, chloronaphthol and the like. The method of preparing conjugates of fluoresces and a protein, such as antibodies, is extensively described in the literature and does not require exemplification here.

Further amplification of the immunohistochemistry signal can be achieved by using combinations of specific binding members, such as antibodies and anti-antibodies, where the anti-antibodies bind to a conserved region of the target antibody probe, particularly where the antibodies are from different species, specific binding ligand-receptor pairs, such as biotin-streptavidin or avidin, or polyvalent ligand and monoclonal antibodies, homologous nucleic acid sequences, and the like. Thus, one effectively builds a sandwich of antibodies (binding members), where the first binding member binds to the cellular component and serves to provide for secondary binding, where the secondary binding may or may not involve a label, and may further provide for tertiary binding where the tertiary binding will provide a label. In this way, the signal can be greatly amplified.

It is understood that a variety of antibodies, including mAb-806, antibodies that bind the same epitope as mAb-806, and antibodies with an epitope specificity to one or more of SEQ ID NO:1-14 may be used for the methods of the invention. These antibodies may be tested for effectiveness in detection by using psoriasis tissue samples and animals models. For example, animals suffering from psoriasis (e.g., mange etc) may be treated with candidate antibodies and the effectiveness of the antibodies may be measured. A successful antibody will significantly lower the amount of psoriasis tissue in the animal. The lowering may be at least about 10%, preferably at least about 20%, 30%, 50%, 70%, or more.

### III. Peptide Vaccines

Another aspect of the invention is directed to a method of treating or immunizing a patient against psoriasis, scleroderma, eczema or atopic dermatitis. Peptides have been successfully used to induce an immune response in animal (See, e.g., U.S. Pat. Nos. 4,601,903; 4,599,231; 4,599,230; and 4,596,792) are known. In the method of the invention, an immunogenic composition comprising an amount of the receptor peptide, or immunogenic fragments thereof and combinations thereof is administered to a patient with psoriasis, scleroderma, eczema or atopic dermatitis. The patient is induced to produce antibodies against the peptides. The produced antibodies, which would have the same specificity against mAb-806, would bind to psoriasis, scleroderma, eczema or atopic dermatitis tissue and cause a regression of the tissue. The peptide may be SEQ ID NO:1-14 or a combination thereof.

In a preferred embodiment, the peptide or immunogenic compositions may be prepared as injectables, as liquid solutions or emulsions. The antigens and immunogenic compositions may be mixed with physiologically acceptable carriers which are compatible therewith. These may include water, saline, dextrose, glycerol, ethanol and combinations thereof. The vaccine may further contain auxiliary substances, such as wetting or emulsifying agents or pH buffering agents, to further enhance their effectiveness. Vaccines may be administered by injection subcutaneously or intramuscularly.

Alternatively, the immunogenic compositions may be formulated and delivered in a manner to evoke an immune response at mucosal surfaces. Thus, the immunogenic composition may be administered to mucosal surfaces by, for example, the nasal or oral (intragastric) routes. Alternatively, other modes of administration including suppositories may be desirable. For suppositories, binders and carriers may include, for example, polyalkylene glycols and triglycerides. Oral formulations may include normally employed excipients, such as pharmaceutical grades of saccharine, cellulose and magnesium carbonate.

Methods for administration, for any of the methods and compositions of the invention, including the peptides and antibodies described anywhere, may include patenteral administration, topical administration and oral administration. Where a peptide is used, the peptide may be administered with an adjuvant to the host animal where the adjuvant and associated antigens are immumostimulatively effective. Delivery modes for any of the methods of the invention and for any of the compositions of the invention may include, without limitation, parenteral administration methods, such as paracancerally, transmucosally, transdermally, intramuscularly, intravenously, intradermally, subcutaneously, intraperitonealy, intraventricularly, intracranially and intratumorally.

The invention may be better understood by reference to the following non-limiting Examples, which are provided as exemplary of the invention. The following examples are presented in order to more fully illustrate the preferred embodiments of the invention and should in no way be construed, however, as limiting the broad scope of the invention.

### EXAMPLES

### Example 1

Immunohistochemical Detection of Psoriasis Tissue Using mAb-806

To determine if we can selectively bind psoriasis samples with the mAb 806, we performed a series of immunohistochemistry analysis of 14 psoriatic tissue samples for wildtype EGFR expression and 806 antigen binding (i.e., staining) using standard methodologies known in the art and described, for example, in Jungbluth et al., PNAS, 100:639-44 (2003).

In summary, all 14 psoriasis showed expression of wildtype EGFR expression (which is also highly positive in normal skin), and 11 of the 14 showed binding to the 806 monoclonal antibody. See FIG. 1. Binding of the 806 monoclonal antibody was confined to the basal layer of the epidermis in psoriatic lesions and was negative in normal skin.

The negative psoriatic lesions had either hyperkertosis or a pustular component. The significance of this is uncertain, and we are extending our samples further. This data is consistent with our model that while EGFR is expressed in all skin tissues, the 806 epitope is only available for antibody binding psoriatic tissue where an autocrine loop is known to be involved in the psoriatic inflammatory process.

This invention may be embodied in other forms or carried out in other ways without departing from the spirit or essential characteristics thereof. The present disclosure is therefore to be considered as in all aspects illustrate and not restrictive, the scope of the invention being indicated by the appended Claims, and all changes which come within the meaning and range of equivalency are intended to be embraced therein.

Various references are cited throughout this Specification, each of which is incorporated herein by reference in its entirety.

What is claimed is:

1. A method for treating a patient with a skin disorder selected from the group consisting of psoriasis, scleroderma, eczema and atopic dermatitis, comprising administering to said patient a therapeutically effective amount of an antibody selected from the group consisting of:
(a) an anti-EGFR antibody selected from mAb-806 antibody or an active fragment thereof;
(b) an antibody capable of binding to the epitope on EGFR that is recognized by mAb-806 antibody;
(c) an antibody with an epitope specificity to one or more peptides selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, and SEQ ID NO:14;
(d) and a combination thereof;
to said patient whereby said skin disorder is treated.

2. The method of claim 1 wherein said mAb-806 antibody comprises the polypeptide of amino acids substantially as set out in either one or more of SEQ ID NO:18, SEQ ID NO:19, SEQ ID NO:20 or SEQ ID NO:21.

3. The method of claim 1 wherein said antibody capable of binding to the epitope on EGFR that is recognized by the mAb-806 antibody comprises one or more polypeptide binding domains each comprising an amino acid sequence substantially similar to a peptide with a sequence selected from the group consisting of GYSITSDFAWN (SEQ ID NO:22), GYISYSGNTRYNPSLK (SEQ ID NO:23), and VTAGRGFPY (SEQ ID NO:24).

4. The method of claim 1 wherein said antibody capable of binding to the epitope on EGFR that is recognized by mAb-806 antibody comprises one or more polypeptide binding domains each comprising an amino acid sequence substantially similar to a peptide with a sequence selected from the group consisting of HSSQDINSNIG (SEQ ID NO:25), HGTNLDD (SEQ ID NO:26), and VQYAQFPWT (SEQ ID NO:27).

5. The method of claim 1, wherein SEQ ID NO:11 has an amino acid sequence C X₁ X₂ X₃ X₄ X₅ X₆ X₇ X₈ X₉ X₁₀ X₁₁ X₁₂ X₁₃ X₁₄ X₁₅ wherein each Xₙ residue can be independently selected as follow:
X₁ is G, P, N or S;
X₂ is A, P, T, S or L;
X₃ is D, H or S;
X₄ is S, Y, T, N or K;
X₅ is Y, Q or M;
X₆ is M or V;
X₇ is E, T or D;
X₈ is A or none;
X₉ is E or K;
X₁₀ is D or N;
X₁₁ is G or A;
X₁₂ is V, I, L or T;
X₁₃ is R, Q or K;
X₁₄ is R, K or M;
X₁₅ is C or none.

6. The method of claim 1, wherein SEQ ID NO:12 has an amino acid sequence C X₁ X₂ X₃ X₄ X₅ X₆ X₇ X₈ X₉ X₁₀ X₁₁ X₁₂ X₁₃ X₁₄ X₁₅ wherein each Xₙ residue can be independently selected as follows:
X₁ is G, P, N, Q, S or T;
X₂ is A, P, T, S, L, M, V, I or P;
X₃ is D, E, H, R, K, S or T;
X₄ is S, Y, F, W, T, N, Q, K or R;
X₅ is Y, F, W, Q, N, M, V, A, L, I or P;
X₆ is M, V, A, L, I or P;
X₇ is E, D, T or S;
X₈ is A, V, L, I, P, M or none;
X₉ is D, E, K or R;
X₁₀ is D, E, N or Q;
X₁₁ is G, A, M, V, L, I or P;
X₁₂ is V, I, L, M, A, P, S or T;
X₁₃ is R, K, H, Q or N;
X₄ is R, K, H, M, A, V, L, I or P;
X₁₅ is C or none.

7. The method of claim 1, wherein SEQ ID NO:13 has an amino acid sequence C X₁ X₂ X₃ X₄ X₅ E X₆ X₇ X₈ X₉ G X₁₀ X₁₁ X₁₂ C wherein each Xₙ residue can be independently selected as follows:
X₁ is G or A
X₂ is A or K
X₃ is D or A
X₄ is S or A
X₅ is Y or A
X₆ is M or A
X₇ is E or A
X₈ is E or A
X₉ is D or A
X₁₀ is V, A or K
X₁₁ is R or A
X₁₂ is K or A.

8. The method of claim 1, wherein SEQ ID NO:14 has an amino acid sequence C X₁ X₂ X₃ X₄ X₅ E X₆ X₇ X₈ D C V R K C wherein each Xₙ residue can be independently selected as follows:
X₁ is G or A
X₂ is A or K
X₃ is D or A
X₄ is S or A
X₅ is Y or A
X₆ is M or A
X₇ is E or A
X₈ is E or A.

9. The method of claim 1 wherein said antibody is a humanized antibody, a single chain antibody, or an engineered antibody.

10. The method of claim 1 wherein said antibody further comprises a detectable label.

11. The method of claim 1 wherein said antibody is conjugated by a chemical bond to one or more moieties selected from the group consisting of a toxin, a chemotherapeutic agent, a radionuclide and a combination thereof.

12. The method of claim 1 wherein the antibody is pegylated.

13. The method of claim 1 wherein the antibody is in the form of an antibody F(ab′)₂, scFv fragment, diabody, triabody or tetrabody.

14. The method of claim 11, wherein the toxin is selected from the group consisting of ricin, abrin, ribonuclease, DNase I, Staphylococcal enterotoxin-A, pokeweed antiviral protein, gelonin, diphtherin toxin, Pseudomonas exotoxin, and Pseudomonas endotoxin.

15. The method of claim 11, wherein the chemotherapeutic agent is selected from the group consisting of taxanes, nitrogen mustards, ethylenimine derivatives, alkyl sulfonates, nitrosoureas, triazenes, folic acid analogs, pyrimidine analogs, purine analogs, vinca alkaloids, antibiotics, enzymes, platinum coordination complexes, substituted urea, and methyl hydrazine derivatives.

16. The method of claim 11, wherein the chemotherapeutic agent is selected from the group consisting of azaribine, bleomycin, bryostatin-1, busulfan, carmustine, chlorambucil, cisplatin, CPT-11, cyclophosphamide, cytarabine, dacarbazine, dactinomycin, daunorubicin, dexamethasone, diethylstilbestrol, doxorubicin, ethinyl estradiol, etoposide, fluorouracil, fluoxymesterone, gemcitabine, hydroxyprogesterone caproate, hydroxyurea, L-asparaginase, leucovorin, lomustine, mechlorethamine, medroprogesterone acetate, megestrol acetate, melphalan, mercaptopurine, methotrexate, methotrexate, mithramycin, mitomycin, mitotane, phenyl butyrate, prednisone, procarbazine, semustine streptozocin, tamoxifen, taxol, testosterone propionate, thalidomide, thioguanine, thiotepa, uracil mustard, vinblastine, and vincristine

17. The method of claim 11, wherein the radionuclide is selected from the group consisting of ²²⁵Ac, ¹¹¹Ag, ⁷²As, ⁷⁷As, ²¹¹At, ¹⁹⁸Au, ¹⁹⁹Au, ²¹²Bi, ²¹³Bi, ⁷⁵Br, ⁷⁶Br, ¹¹C, ⁵⁵Co, ⁶²Cu, ⁶⁴CU, ⁶⁷CU, ⁶⁷CU, ¹⁶⁶Dy, ¹⁶⁹Er, ¹⁸F, ⁵²Fe, ⁵⁹Fe, ⁶⁷Ga, ⁶⁸Ga, ¹⁵⁴⁻¹⁵⁸Gd, ¹⁶⁶Ho, ¹²⁰I, ¹²¹I, ¹²³I, ¹²⁴I, ¹²⁵I, ¹³¹I, ¹³¹I, ¹¹⁰In, ¹¹¹In, ¹⁹⁴Ir, ¹⁷⁷Lu, ⁵¹Mn, ⁵²Mn, ⁹⁹Mo, ¹³N, ¹⁵O, ³²P, ³³P, ²¹¹Pb, ²¹²Pb, ¹⁰⁹Pd, ¹⁴⁹Pm, ¹⁴²Pr, ¹⁴³Pr, ²²³Ra, ⁸²Rb, ¹⁸⁶Re, 188Re, ¹⁸⁹Re, ¹⁰⁵Rh, ⁴⁷Sc, ⁷⁵Se, ¹⁵³Sm, ⁸³Sr, ⁸⁹Sr, ¹⁶¹Tb, ⁹⁴Tc, ⁹⁹Tc, ⁸⁶Y, ⁹⁰Y and ⁸⁹Zr.

18. The method of claim 11, wherein said administration comprises administrating a radionuclide of between 10 and 40 mCi.

19. The method of claim 11, wherein said administration comprises administrating a radionuclide of between 20 and 30 mCi.

20. The method of claim 1 wherein said treatment reduces at least one symptom of said skin disorder in said patient.

21. The method of claim 20 wherein said antibody blocks an EGFR to ligand interaction in said psoriatic tissue, scleroderma tissue, eczema tissue, or atopic dermatitis tissue.

22. The method of claim 20, wherein said antibody causes a regression of EGFR+ cells in said psoriatic tissue, scleroderma tissue, eczema tissue, or atopic dermatitis tissue.

23. The method of claim 22 wherein said regression of EGFR+ cells causes a regression of a psoriasis phenotype, a scleroderma phenotype, an eczema phenotype or an atopic dermatitis phenotype in said patient.

24. The method of claim 23 wherein said psoriasis phenotype is selected from the group consisting of plaque phenotype, guttate phenotype, inverse phenotype, pustular phenotype and erythrodermic phenotype.

25. The method of claim 1, wherein the patient is a human.

26. A method for detecting a skin disorder in a patient selected from the group consisting of psoriasis, scleroderma, eczema and atopic dermatitis, comprising:
a) administering an antibody selected from the group consisting of a mAb-806 antibody or active fragment thereof; an antibody capable of binding to the epitope on EGFR that is recognized by mAb-806 antibody; an antibody with an epitope specificity to one or more of the peptides SEQ ID NO: 1-14; and a combination thereof to said patient; and
b) detecting specific binding of the antibody to an EGFR of said psoriatic tissue, scleroderma tissue, eczema tissue or atopic dermatitis tissue.

27. The method of claim 26, wherein the antibody comprises a detectable label.

28. The method of claim 27 wherein the detectable label is selected from the group consisting of a fluorescent compound, a chemiluminescent compound, and a bioluminescent compound, and a radionuclide.

29. The method of claim 28 wherein said fluorescent compound is selected from the group consisting of fluorescein isothiocyanate, rhodamine, phycoerytherin, phycocyanin, allophycocyanin, o-phthaldehyde and fluorescamine.

30. The method of claim 28 wherein said chemiluminescent compound is selected from the group consisting of luminol, isoluminol, an aromatic acridinium ester, an imidazole, an acridinium salt and an oxalate ester.

31. The method of claim 28 wherein said bioluminescent compound is selected from the group consisting of luciferin, luciferase and aequorin.

32. The method of claim 27 wherein detecting specific binding of the antibody comprises detecting a label from said detectably labeled antibody in said tissue.

33. The method of claim 26 wherein detecting specific binding of the antibody comprises:
a) obtaining a sample of the tissue from the patient; and
b) detecting specific binding of the antibody to said sample.

34. The method of claim 26, wherein detecting specific binding of the antibody is performed using an in vivo imaging method.

35. The method of claim 34, wherein the in vivo imaging method is selected from the group consisting of radionuclide imaging, positron emission tomography, computerized axial tomography, and magnetic resonance imaging.

36. The method of claim 26, wherein the patient is a human.

37. A test kit for detecting a skin disorder in a patient selected from the group consisting of psoriasis, scleroderma, eczema and atopic dermatitis, comprising:
A. a predetermined amount of an antibody selected from the group consisting of a mAb-806 antibody or active fragment thereof; an antibody capable of binding to the epitope on EGFR that is recognized by mAb-806 antibody; an antibody with an epitope specificity to one or more of the peptides SEQ ID NO: 1-14; and a combination thereof;
B. a predetermined amount of a specific binding partner of said antibody;
C. other reagents; and
D. directions for use of said kit;
wherein either said antibody or said specific binding partner are detectably labelled.

38. The test kit of claim 37, wherein the specific binding partner is selected from one or more of the peptides SEQ ID NO: 1-14.

39. A method of treating a patient with a skin disorder selected from the group consisting of psoriatis tissue, scleroderma tissue, eczema tissue and atopic dermatitis tissue, comprising the step of administering an peptide selected from any of SEQ ID NO:1-14 or an immunogenic fragment thereof to said patient whereby antibodies immunoreactive with the peptide or immunogenic fragment thereof is produced and wherein said antibodies bind an epitope present on said psoriatic tissue, scleroderma tissue, eczema tissue or atopic dermatitis tissue whereby said skin disorder is treated.

40. The method of claim 39, wherein said peptide is parenterally administered in a dosage of 20 to 1500 milligrams peptide per dose.

41. The method of claim 39, wherein said peptide is parenterally administered in a dosage of 20 to 500 milligrams protein per dose.

42. The method of claim 39, wherein said peptide is parenterally administered in a dosage of 20 to 100 milligrams protein per dose.

43. The method of claim 39, wherein the patient is a human.
